(19) United States
(12) Patent Application Publication
(10) Pub. No.: US 2005/0143599 A1
(43) Pub. Date: Jun. 30, 2005

(54) Process for preparing monoesters

(76) Inventors: Markus A. Wicki, London (CA); Kent E. Nielsen, Dorchester (CA)

Correspondence Address: 3M INNOVATIVE PROPERTIES COMPANY, PO BOX 33427, ST. PAUL, MN, 55133-3427, US

(21) Appl. No.: 10/748,939

(22) Filed: Dec. 30, 2003

(51) Int. Cl. C07C 67/08; C07C 67/14

(52) U.S. Cl. 560/261; 560/263

(57) ABSTRACT

A process for preparing monoesters comprises the step of reacting at least one diol with at least one carboxylic acid in a biphasic solvent system, the carboxylic acid being sufficiently water soluble to allow esterification to occur, and the biphasic solvent system comprising water and at least one aprotic solvent in which the resulting monoester has greater solubility than in water.

### FIELD

This invention relates to a process for preparing monoesters such as, for example, monoacetates from diols.

### BACKGROUND

Insect pests such as, for example, leafrollers, tomato pinworms, oriental fruit moths, and codling moths can cause significant damage and economic loss in the production of tree fruits, vine and nut crops, and various other crops. In the past, such insect pests were typically controlled with pesticides such as, for example, organophosphate pesticides. Due to regulatory and environmental pressures, however, insect pest control is moving away from exclusive reliance on pesticides. As a result, alternative crop protection strategies such as insect mating disruption technology have steadily increased in general acceptance.

Insect mating disruption is an important component of the modern approach to pest regulation known as integrated pest management, which combines biological, cultural, physical, and chemical techniques to regulate pest populations while minimizing cost and environmental disturbances. The typical mating disruption technique confuses male insects with pheromones from the natural chemical blends of conspecific females. Sources of sex pheromone are placed in a crop or environment at concentrations sufficient to mask the presence of females. Decreasing or delaying the mating of the moths thus decreases the population of the next generation of larvae, as well as the potential for future crop or environmental damage.

Numerous approaches to preparing pheromone compounds for use in insect mating disruption have been investigated. Several commercially promising approaches focus on the use of readily available symmetric diols as starting materials. For example, one of the two hydroxy groups of a symmetric diol can be acetylated to form a monoacetate (see, for example, Babler et al., Tetrahedron Lett., 22, 1971 (1979)). The monoacetate can then be readily converted into pheromone compounds.

The economic value of such sequences in a commercial setting, however, is highly dependent upon the selectivity of the monoacetylation. By reacting one equivalent of an acetylating agent with a symmetric diol under standard conditions, a mixture of diacetate, monoacetate, and unreacted diol in a statistical distribution of 1:2:1 would be obtained. Therefore, approaches have been developed to steer the selectivity of the acetylation of symmetric diols to favor the monoacetate product.

Many of these approaches, however, are not well suited for use on a commercial manufacturing scale. Some approaches, for example, require specialized equipment such as continuous extraction apparatuses (see, for example, Babler et al., Tetrahedron Lett., 22, 1971 (1979)). Other approaches require large quantities of relatively expensive reagents such as silica gel (see, for example, Nishiguchi, J. Am. Chem. Soc., 111, 9102 (1989), and Ogawa et al., J. Chem. Soc. Chem. Commun. 495 (1998)) or catalysts such as Cornybacterium oxydans (see, for example, EP 0 280 232 B1) and zeolites (see, for example, EP 0 627404 B1), which could cause technical difficulties and economic burden when used on a commercial manufacturing scale.

### SUMMARY

In view of the foregoing, we recognize that there is a need for an improved process for selectively transforming symmetric diols into monoacetates. Furthermore, we recognize that in order for such a process to be commercially viable, it should eliminate the necessity for specialized equipment and expensive reagents and/or catalysts.

Briefly, the present invention provides a process for preparing monoesters from diols. The process comprises the step of reacting at least one diol (preferably, a symmetric diol) with at least one carboxylic acid in a biphasic (two-phase) solvent system, the carboxylic acid being sufficiently water soluble to allow esterification to occur, and the biphasic solvent system comprising water and at least one aprotic solvent in which the resulting monoester has greater solubility than in water.

Surprisingly, it has been discovered that the process of the invention can be used to selectively prepare monoesters such as, for example, monoacetates from symmetric diols. The process does not require specialized equipment, and it can be carried out using inexpensive, commercially available reagents.

The process of the invention therefore meets the need in the art for an improved process for selectively transforming symmetric diols into monoacetates.

Furthermore, while the primary benefit of the process of the invention is an improvement in the selectivity of the monoacetylation of symmetric diols, improvements in the yield and selectivity of the monoesterification of asymmetric diols can also be achieved using the process of the invention.

### DETAILED DESCRIPTION

Diol Starting Compounds

Diols, or dihydric alcohols, are alcohols containing two hydroxyl groups connected to different carbon atoms. Any diol can be used as a starting compound for carrying out the process of the invention. Preferably, diols having less than about 14 carbon atoms are used; more preferably, diols having less than about 12 carbon atoms.

Diols that are useful in carrying out the process of the invention can comprise heteroatoms (that is, the carbon backbone of the diol can be interrupted by heteroatoms such as nitrogen, phosphorus, sulfur, iodine, or oxygen) or substituents. They can also be substituted with substituents such as, for example, halogens, alkyl groups, alkenyl groups, alkynyl groups, alkoxy groups, aryl groups, aryloxy groups, nitro groups, and the like.

Diols that are useful in carrying out the process of the invention include diprimary, disecondary, and ditertiary alcohols (preferably, diprimary or disecondary alcohols; more preferably, diprimary alcohols).

Representative examples of diprimary alcohols that are useful starting compounds include ethanediol, 1,3-propanediol, 1,4-butanediol, 1,5-pentanediol, 1,6-hexanediol, 1,7-heptanediol, 1,8-octanediol, 1,9-nonanediol, 1,10-decanediol, 1,11-undecanediol, 1,12-dodecanediol, 4-methyl-1,8-octanediol, 4-ethyl-1,8-octanediol, 1,11-undec-5-enediol, and the like, and mixtures thereof. Preferred diprimary alcohols include, for example, 1,8-octanediol, 1,9-nonanediol, 1,10-decanediol, 1,11-undecanediol, and mixtures thereof. More preferred diprimary alcohols include, for example, 1,8-octanediol, 1,9-nonanediol, 1,11-undecanediol, and mixtures thereof.

Representative examples of disecondary alcohols that are useful starting compounds include 1,4-cyclohexanediol, 2,7-nonanediol, decahydro-naphthalene-2,6-diol, 1-[4-(1-hydroxyethyl)-phenyl]-ethanol, and the like, and mixtures thereof. A preferred disecondary alcohol is, for example, 1,4-cyclohexanediol.

Representative examples of ditertiary alcohols that are useful starting compounds include 1,1,7,7-tetramethylheptane-1,7-diol, 2,6-dimethyl-decahydro-naphthalene-2,6-diol, 1,1,10,10-tetramethyldec-5-ene-1,10-diol, and the like, and mixtures thereof.

Preferably, diols used in carrying out the process of the invention are symmetrical or comprise two hydroxyl groups having substantially equal reactivity.

Symmetric diols are diols that meet the three elements of symmetry as defined by Hawley's Condensed Chemical Dictionary 1067 (1997). First, they have a center of symmetry, around which the constituent atoms are located in an ordered arrangement. There is only one such center in the molecule, which may or may not be an atom. Second, they have a plane of symmetry, which divides the molecule into mirror-image segments. Third, they have axes of symmetry, which can be represented by lines passing through the center of symmetry. If the molecule is rotated, it will have the same position in space more than once in a complete 360° turn.

Representative examples of symmetric diols include ethanediol, 1,3-propanediol, 1,4-butanediol, 1,5-pentanediol, 1,6-hexanediol, 1,7-heptanediol, 1,8-octanediol, 1,9-nonanediol, 1,10-decanediol, 1,11-undecanediol, 1,12-dodecanediol, 1,4-cyclohexanediol, 4-methyl-1,7-heptanediol, and 5,5-dimethyl-1,9-nonanediol.

Diols comprising two hydroxyl groups having substantially equal reactivity are those for which a reactant would not preferentially react with one hydroxyl group over the other. Such diols can be symmetrical or asymmetrical. If the diol is asymmetrical, the two hydroxyl groups are typically sufficiently isolated from asymmetry-causing substituents such that their reactivities are essentially unaffected by the substituents. For example, the two hydroxyl groups in the asymmetrical diol 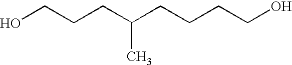
4-methyl-1,8-octanediol
   have substantially equal reactivities because they are each sufficiently isolated from the methyl substituent.

Other representative examples of diols comprising two hydroxyl groups having substantially equal reactivity include 1,11-undec-5-enediol, 4-ethyl-1,8-octanediol, and 2,7-nonanediol.

Carboxylic Acid Starting Compounds

Carboxylic acid starting compounds useful in the process of the invention include those that are sufficiently water soluble to affect esterification. Experimental results in carrying out the process of the invention appear to be consistent with esterification taking place in the water phase of the biphasic solvent system. Therefore, useful carboxylic acid starting compounds include those that are sufficiently water soluble that the concentration of carboxylic acid in the water phase is high enough for esterification to occur. If the carboxylic acid is not sufficiently water soluble, esterification does not appear to occur (that is, the diol appears to remain unreacted).

Preferably, carboxylic acid starting compounds have a solubility in water of at least about 20% by weight (based on total weight of carboxylic acid and water) at 20° C. (preferably, at least about 50% at 20° C.; more preferably, about 100% at 20° C.). Such carboxylic acids generally contain about five carbon atoms or less. Representative examples of suitable carboxylic acid starting compounds include formic acid, acetic acid, trifluoroacetic acid, n-butyric acid, isobutyric acid, pyruvic acid, propionic acid, and mixtures thereof.

Preferably, carboxylic acid starting compounds have solubility in water greater than or equal to that of isobutyric acid. More preferably, carboxylic acid starting compounds are selected from the group consisting of formic acid, acetic acid, trifluoroacetic acid, n-butyric acid, pyruvic acid, propionic acid, and mixtures thereof. Even more preferably, carboxylic acid starting compounds are selected from the group consisting of formic acid, acetic acid, and mixtures thereof. Most preferably, the carboxylic acid starting compound is acetic acid.

Preparation of Monoesters

The step of reacting at least one diol with at least one carboxylic acid can be carried out by combining the diol and the carboxylic acid in a biphasic solvent system comprising water and at least one aprotic solvent. Experimental results in carrying out the process of the invention appear to be consistent with the esterification taking place in the water phase of the biphasic solvent system, and the resulting monoester partitioning out of the water phase and becoming isolated in the aprotic solvent phase. Therefore, useful aprotic solvents include those in which the resulting monoester is more soluble than in water. Useful aprotic solvents form a biphasic (two-phase) system with water.

Preferably, the aprotic solvent has a Snyder polarity index between about 1.5 and about 3.5 (preferably, between about 2.0 and about 3.0). The Snyder polarity index is a relative measure of the degree of interaction of the solvent with various polar test solutes (see, Snyder, J. Chromatogr., 92, 223 (1974)).

Useful aprotic solvents include, for example, methylene chloride, carbon tetrachloride, toluene, benzene, chlorobenzene, ethylbenzene, xylenes, trifluorotoluene, dichlorobenzene, methyl tert-butyl ether (MTBE), diethyl ether, diisopropyl ether, dibutyl ether, benzyl ether, and the like, and mixtures thereof.

Preferably, the aprotic solvent is an aromatic or ether solvent. More preferably, the aprotic solvent is selected from the group consisting of toluene, benzene, chlorobenzene, ethylbenzene, xylenes, trifluorotoluene, dichlorobenzene, methyl tert-butyl ether, diethyl ether, diisopropyl ether, dibutyl ether, and the like, and mixtures thereof. Even more preferably, the aprotic solvent is an aromatic solvent. Most preferably, the aprotic solvent is toluene.

Preferably, the reaction mixture is agitated. The reaction can generally be carried out at a temperature at which the mixture refluxes, typically between about 30° C. and about 120° C.

Optionally, the reaction can be carried out in the presence of an acid catalyst. Useful acid catalysts include acids that are stronger acids than the carboxylic acid starting compound utilized, and are soluble in water. Representative examples of useful acid catalysts include sulfuric acid, nitric acid, hydrochloric acid, hydrobromic acid, hydriodic acid, trifluoroacetic acid, and the like, and mixtures thereof. Preferred acid catalysts include, for example, sulfuric acid, nitric acid, hydrochloric acid, and mixtures thereof.

After the reaction is carried out, the water phase can be separated from the solvent phase. The solvent can then be evaporated to yield the monoester product, along with relatively small amounts of diester and unreacted diol (for example, typically less than about 20% diester (preferably, less than about 10%; more preferably less than about 5%); and typically less than about 10% unreacted diol (preferably, less than about 5%; more preferably, less than about 2%)).

Monoesters

The above-described diols and carboxylic acid starting compounds can be reacted to form the corresponding monoesters. Monoesters that can be prepared using the process of the invention include monoformates, monoacetates, monotrifluoroacetates, monobutyrates, and monopropionates, such as, for example, acetoxyethanol, 3-acetoxypropan-1-ol, 4-acetoxybutan-1-ol, 5-acetoxypentan-1-ol, 6-acetoxyhexan-1-ol, 7-acetoxyheptan-1-ol, 8-acetoxyoctan-1-ol, 9-acetoxynonan-1-ol, 10-acetoxydecan-1-ol, 11-acetoxyundecan-1-ol, 12-acetoxydodecan-1-ol, 4-acetoxycyclohexan-1-ol, 1-acetoxydec-5-en-10-ol, 2-acetoxydecahydro-naphthalene-6-ol, 1-[4-(1-acetoxyethyl)-phenyl]-ethanol, 11-trifluoroacetoxyundecan-1-ol, and the like.

Preferred monoesters include monoacetates such as, for example, acetoxyethanol, 3-acetoxypropan-1-ol, 4-acetoxybutan-1-ol, 5-acetoxypentan-1-ol, 6-acetoxyhexan-1-ol, 7-acetoxyheptan-1-ol, 8-acetoxyoctan-1-ol, 9-acetoxynonan-1-ol, 10-acetoxydecan-1-ol, 11-acetoxyundecan-1-ol, and 12-acetoxydodecan-1-ol. Preferred monoacetates include, for example, 8-acetoxyoctan-1-ol, 9-acetoxynonan-1-ol, 10-acetoxydecan-1-ol, ll-acetoxyundecan-1-ol, 12-acetoxydodecan-1-ol, and 4-acetoxycyclohexan-1-ol. More preferred monoacetates include, for example, 8-acetoxyoctan-1-ol, 9-acetoxynonan-1-ol, and 11-acetoxyundecan-1-ol.

Use of Monoester Products

Monoesters prepared using the process of the invention can be useful as starting compounds in processes for preparing ester group-containing olefinic compounds such as, for example, flavors, fragrances, and semiochemicals. As used herein, “semiochemical” means a chemical that conveys a signal from one organism to another, for example, in such a way as to modify the behavior of the recipient organism (including, for example, allomones, kairomones, synomones, and pheromones, which can have, for example, arrestant, attractant, repellent, deterrent, or stimulant properties).

For example, ester group-containing olefinic compounds can be prepared from monoesters using a two-step procedure comprising an oxidation step and an olefination step.

Oxidation of the remaining hydroxyl group of a monoester prepared using the process of the invention can be carried out to form the corresponding carbonyl-containing compound, which is typically an aldehyde or a ketone. Useful oxidizing agents include, for example, o-iodoxybenzoic acid (IBX), 2,2,6,6-tetramethyl-1-piperidinyloxy and derivatives (TEMPO), pyridinium chlorochromate, 1,1,1-tris(acetyloxy)-1,1-dihydro-1,2-benziodoxol-3-(1H)-one (Dess-Martin), dimethyl sulfoxide, sodium nitrite, sulfur trioxide/amine complex, and the like, and mixtures thereof.

Preferably, the reaction is carried out in the presence of a solvent such as an ether, halogenated solvent, or sulfoxide. Examples of useful solvents include, for example, dimethyl sulfoxide (DMSO), methylene chloride, methyl tert-butyl ether, and the like, and mixtures thereof. If desired, the reaction mixture can be agitated. The reaction can be carried out at a temperature between about −70° C. and about 60° C. (preferably, at room temperature).

Next, the carbonyl-containing compound can be brought together and allowed to react with an alkylidene phosphorane to form the corresponding ester group-containing olefinic compound (see, for example, Babler et al., Tetrahedron Lett., 22, 1971 (1979), and Applicants' copending application U.S. Ser. No. 10/692358, filed Oct. 23, 2003). The reaction is generally carried out under an inert gas atmosphere (that is, in the absence of oxygen), and under anhydrous conditions. Preferably, the reaction mixture is agitated.

Representative examples of useful alkylidene phosphoranes include, for example, butylidenetriphenylphosphorane, propylidenetriphenylphosphorane, butenylidenetri(n-butyl)phosphorane, nonylidenetri(n-nonyl)phosphorane, 2-pentenylidenetri(n-butyl)phosphorane, propylidenetri(n-propyl)phosphorane, butylidenetri(n-butyl)phosphorane, pentylidene n-pentyldiphenylphosphorane, propylidenediphenylphosphine oxide anion, butenylidenediphenylphosphine oxide anion, nonylidenediphenylphosphine oxide anion, 2-pentenylidenediphenylphosphine oxide anion, butenylidene diethylphosphonate anion, nonylidene diethylphosphonate anion, and the like, and mixtures thereof.

If desired, olefinic esters prepared using the above-described procedure can be hydrolyzed to form the corresponding alcohols. Olefinic acetates prepared using the above-described procedure can be oxidized to form the corresponding aldehydes. Useful oxidizing agents include, for example, nitrogen oxides such as pyridine-N-oxide and trimethylamine-N-oxide.

Representative flavor compounds that can be prepared using the above-described procedure with monoester starting compounds prepared using the process of the invention include, for example, Z-3-octen-1-ol (watermelon, cucumber flavor), Z-4-hepten-1-al (used in cream and butter), Z-3-hexenyl pyruvate (celery flavor), E-3-octen-2-ol (mushroom, tomato flavor), and E,E-2,4-hexadien-1-ol (pineapple flavor).

Representative fragrances that can be prepared using the above-described procedure with monoester starting compounds prepared using the process of the invention include, for example, Z-3-hexenyl acetate (sharp fruity green), Z-3-hexenyl methylbutyrate (green apples), E-2-heptenyl acetate (berry note), and E-2-octenyl butyrate.

Semiochemicals prepared using the above-described process with monoester starting compounds prepared using the process of the invention can be useful, for example, in pheromones.

Representative pheromone compounds that can be prepared using the above-described procedure with monoester starting compounds prepared using the process of the invention include, for example, 11-tetradecenal (for Eastern Spruce Budworm pheromone), 10-nonadecenal (for Yellow Headed Spruce Sawfly pheromone), 8,10-dodecadienol (for Codling Moth pheromone), 8,10-dodecadienol acetate (for Pea Moth), 11-tetradecenol (for Tufted Apple Budmoth pheromone), 11-tetradecenyl acetate (for Tufted Apple Budmoth pheromone, Sparganothis Fruitworm pheromone, Leafroller pheromone, and Blackheaded Fireworm pheromone), 9-dodecenyl acetate (for Grape Berry Moth pheromone and Tea Tortrix pheromone), 4-tridecenyl acetate (for Tomato Pinworm pheromone), 7,11-hexadecadienyl acetate (for Pink Cotton Bollworm pheromone), 8-dodecenyl acetate (for Oriental Fruit Moth pheromone and Citrus Fruit Moth pheromone), and 3,13-octadecadienyl acetate (for Peach Tree Borer pheromone and Lesser Peach Tree Borer pheromone).

Preferred pheromone compounds that can be prepared using the above-described procedure with monoester starting compounds prepared using the process of the invention are alcohols and acetates such as, for example, 11-tetradecenyl acetate, 9-dodecenyl acetate, 4-tridecenyl acetate, 7,11-hexadecadienyl acetate, 8-dodecenyl acetate, 3,13-octadecadienyl acetate, 8,10-dodecadienol, and 8,10-dodecadienyl acetate.

Pheromone compounds prepared as described above can be used in mating disruption products. For example, the pheromone compounds can be microencapsulated and used in sprayable compositions to control insect pest activity.

### EXAMPLES

Objects and advantages of this invention are further illustrated by the following examples, but the particular materials and amounts thereof recited in these examples, as well as other conditions and details, should not be construed to unduly limit this invention.

Unless otherwise specified, all starting materials were obtained from Sigma-Aldrich Canada.

### Example 1

In a 250 mL round bottom flask, 1,8-octanediol (5.01 g, 34.3 mmol) was suspended in toluene (100 mL). Water (3.5 mL) and acetic acid (2.05 mL, 35.8 mmol) were added, and the reaction mixture was heated to reflux temperature. After 16 hours of reflux, the mixture was allowed to cool to room temperature. The aqueous phase was separated and the organic phase was washed with saturated aqueous sodium bicarbonate solution (50 mL), water (50 mL), and brine (50 mL) and dried (sodium sulfate). After filtration, the solvent was evaporated and the resulting residue was dried. The product was obtained as a colourless liquid (781 mg, 12% yield) with the following composition determined by gas chromatography-mass spectrometry (GC-MS) (Varian 3800/Saturn 2000): diol (7.1%), monoacetate (88.2%), and diacetate (4.7%).

### Example 2

In a 250 mL round bottom flask, 1,8-octanediol (5.00 g, 34.2 mmol) was suspended in toluene (100 mL). Water (3.5 mL), glacial acetic acid (2.05 mL, 35.8 mmol), and concentrated sulfuric acid (137 mg, 1.40 mmol) were added, and the reaction mixture was heated to reflux temperature. After 48 hours of reflux, the mixture was allowed to cool to room temperature. The aqueous phase was separated and the organic phase was washed with saturated aqueous sodium bicarbonate solution (50 mL), water (50 mL), and brine (50 mL) and dried (magnesium sulfate). After filtration, the solvent was evaporated and the resulting residue was dried. The product was obtained as a colourless liquid (5.32 g, 83% yield) with the following composition determined by GC-MS (Varian 3800/Saturn 2000): diol (1.3%), monoacetate (96.8%), and diacetate (1.9%).

### Example 3

In a 250 mL round bottom flask, 1,8-octanediol (4.99 g, 34.1 mmol) was suspended in toluene (100 mL). Water (3.5 mL), acetic acid (2.05 mL, 35.8 mmol), and aqueous hydrochloric acid (36%, 0.12 mL, 1.41 mmol) were added, and the reaction mixture was heated to reflux temperature. After 16 hours of reflux, the mixture was allowed to cool to room temperature. The aqueous phase was separated and the organic phase was washed with saturated aqueous sodium bicarbonate solution (50 mL), water (50 mL), and brine (50 mL) and dried (sodium sulfate). After filtration, the solvent was evaporated and the resulting residue was dried. The product was obtained as a colourless liquid (4.92 g, 77% yield) with the following composition determined by GC-MS (Varian 3800/Saturn 2000): diol (4.3%), monoacetate (87.8%), and diacetate (7.9%).

### Example 4

In a 250 mL round bottom flask, 1,8-octanediol (5.00 g, 34.2 mmol) was suspended in toluene (100 mL). Water (3.5 mL), acetic acid (2.05 mL, 35.8 mmol), and aqueous nitric acid (70%, 0.09 mL, 1.40 mmol) were added, and the reaction mixture was heated to reflux temperature. After 16 hours of reflux, the mixture was allowed to cool to room temperature. The aqueous phase was separated and the organic phase was washed with saturated aqueous sodium bicarbonate solution (50 mL), water (50 mL), and brine (50 mL) and dried (sodium sulfate). After filtration, the solvent was evaporated and the resulting residue was dried. The product was obtained as a colourless liquid (5.17 g, 80% yield) with the following composition determined by GC-MS (Varian 3800/Saturn 2000): diol (3.2%), monoacetate (86.0%), and diacetate (10.8%).

### Comparative Example 1

In a 250 mL round bottom flask, 1,8-octanediol (4.99 g, 34.1 mmol) was suspended in cyclohexane (100 mL). Water (3.5 mL), acetic acid (2.05 mL, 35.8 mmol), and concentrated sulfuric acid (137 mg, 1.40 mmol) were added, and the reaction mixture was heated to reflux temperature. After 16.5 hours of reflux, the mixture was allowed to cool to room temperature. The aqueous phase was separated and the organic phase was washed with saturated aqueous sodium bicarbonate solution (50 mL), water (50 mL), and brine (50 mL) and dried (sodium sulfate). After filtration, the solvent was evaporated and the resulting residue was dried. The product was obtained as a colourless liquid (2.07 g).

The aqueous phase was extracted with heptane (3×25 mL). To the three combined organic phases was added ethyl acetate until a homogeneous solution was obtained. The organic phase was washed with saturated aqueous sodium bicarbonate solution (25 mL), water (25 mL), and brine (25 mL) and dried (sodium sulfate). After filtration, the solvent was evaporated and the resulting residue was dried. The product was obtained as a yellowish liquid (2.48 g).

The colourless liquid and the yellowish liquid were combined to yield the product as a slightly yellowish liquid (4.36 g, 68% yield) with the following composition determined by GC-MS (Varian 3800/Saturn 2000): diol (9.5%), monoacetate (53.4%), and diacetate (37.1%).

### Comparative Example 2

In a 250 mL round bottom flask, 1,8-octanediol (4.99 g, 34.1 mmol) was suspended in toluene (100 mL). Acetic acid (2.05 mL, 35.8 mmol) and concentrated sulfuric acid (137 mg, 1.40 mmol) were added, and the reaction mixture was heated to reflux temperature. After 16 hours of reflux, the mixture was allowed to cool to room temperature. The reaction mixture was washed with saturated aqueous sodium bicarbonate solution (50 mL), water (50 mL), and brine (50 mL) and dried (sodium sulfate). After filtration, the solvent was evaporated and the resulting residue was dried. The product was obtained as a yellowish liquid (5.43 g, 85% yield) with the following composition determined by GC-MS (Varian 3800/Saturn 2000): diol (0.0%), monoacetate (50.0%), and diacetate (50.0%).

### Example 5

In a 250 mL round bottom flask, 1,8-octanediol (5.01 g, 34.3 mmol) was suspended in toluene (100 mL). Water (3.5 mL), formic acid (1.35 mL, 35.8 mmol), and concentrated sulfuric acid (137 mg, 1.40 mmol) were added, and the reaction mixture was heated to reflux temperature. After one hour of reflux, the mixture was allowed to cool to room temperature. The aqueous phase was separated and the organic phase was washed with saturated aqueous sodium bicarbonate solution (50 mL), water (50 mL), and brine (50 mL) and dried (sodium sulfate). After filtration, the solvent was evaporated and the resulting residue was dried. The product was obtained as a colourless liquid (4.54 g, 76% yield) with the following composition determined by GC-MS (Varian 3800/Saturn 2000): diol (6.7%), monoformate (84.1%), and diformate (9.2%).

### Comparative Example 3

In a 250 mL round bottom flask, 1,8-octanediol (5.00 g, 34.2 mmol) was suspended in toluene (100 mL). Water (3.5 mL), benzoic acid (4.37 g, 35.8 mmol), and concentrated sulfuric acid (137 mg, 1.40 mmol) were added, and the reaction mixture was heated to reflux temperature. After 40 hours of reflux, GC-MS analysis (Varian 3800/Saturn 2000) showed that essentially no reaction had occurred. Only unreacted diol was detected.

### Comparative Example 4

In a 250 mL round bottom flask, 1,8-octanediol (4.99 g, 34.1 mmol) was suspended in toluene (100 mL). Water (3.5 mL), isovaleric acid (4.0 mL, 36.2 mmol), and concentrated sulfuric acid (137 mg, 1.40 mmol) were added, and the reaction mixture was heated to reflux temperature. After 13 hours of reflux, GC-MS analysis (Varian 3800/Saturn 2000) showed that essentially no reaction had occurred. Only unreacted diol was detected.

### Example 6

In a 250 mL round bottom flask, 1,9-nonanediol (5.00 g, 31 mmol) was suspended in toluene (100 mL). Water (3.5 mL), glacial acetic acid (1.9 mL, 33 mmol), and concentrated sulfuric acid (137 mg, 1.40 mmol) were added, and the reaction mixture was heated to reflux temperature. After 48 hours of reflux, the mixture was allowed to cool to room temperature. The aqueous phase was separated and the organic phase was washed with saturated aqueous sodium bicarbonate solution (50 mL), water (50 mL), and brine (50 mL) and dried (sodium sulfate). After filtration, the solvent was evaporated and the resulting residue was dried. The residue (5.24 g) showed the following composition determined by GC-MS (Varian 3800/Saturn 2000): diol (6.0%), monoacetate (90.6%), and diacetate (3.4%).

Further purification was achieved by washing the residue with water. The residue was dissolved in toluene (50 mL), washed with water (3×50 mL) and brine (50 mL), and dried (sodium sulfate). Evaporation of the solvent gave the product (4.57 g, 72% yield) as a colourless liquid with the following composition determined by GC-MS (Varian 3800/Saturn 2000): diol (1.8%), monoacetate (94.3%), and diacetate (3.9%).

### Example 7

In a 250 mL round bottom flask, 1,10-decanediol (5.07 g, 29 mmol) was suspended in toluene (100 mL). Glacial acetic acid (17.41 g, 290 mmol), water (3.5 mL), and concentrated sulfuric acid (137 mg, 1.40 mmol) were added, and the reaction mixture was heated to reflux temperature. The progress of the reaction was monitored by GC-MS. After 3 hours of reflux, the flask was removed from the heating bath and allowed to cool to room temperature. The contents of the flask were transferred to a separatory funnel and washed with water (25 mL), 10% sodium bicarbonate (2×25 mL), and brine (25 mL). The organic phase was dried over sodium sulfate, filtered, and the solvent was removed under reduced pressure to provide 6.12 g (98% yield) of a water-clear liquid, which crystallized on standing at room temperature for two days. Analysis of the final product by GC-MS (Varian 3800/Saturn 2000) confirmed the product composition as 75% monoacetate, 7% diol, and 18% diacetate.

### Example 8

In a 250 mL round bottom flask, 1,11-undecanediol (2.62 g, 14 mmol) was suspended in toluene (50 mL). Glacial acetic acid (8.41 g, 140 mmol), water (2.5 mL), and concentrated sulfuric acid (82 mg, 0.84 mmol) were added, and the reaction mixture was heated to reflux temperature. The progress of the reaction was monitored by GC-MS. After 4 hours of reflux, the flask was removed from the heating bath and allowed to cool to room temperature. The contents of the flask were transferred to a separatory funnel and washed with water (25 mL), 10% sodium bicarbonate (2×25 mL), water (25 mL), and brine (25 mL). The organic phase was dried over sodium sulfate, filtered, and the solvent was removed under reduced pressure to yield 3.08 g (96% yield) of a water-clear liquid, which crystallized on standing at room temperature overnight. Analysis of the final product by gas chromatography-flame ionization detector (GC-FID) (Varian 3600) confirmed the product composition as 73% monoacetate, 14% diol, and 13% diacetate.

### Example 9

In a 250 mL round bottom flask, 1,4-cyclohexanediol (3.98 g, 34.3 mmol) was suspended in toluene (100 mL). Water (3.5 mL), acetic acid (2.05 mL, 35.8 mmol), and concentrated sulfuric acid (137 mg, 1.40 mmol) were added, and the reaction mixture was heated to reflux temperature. After 4 hours of reflux, the mixture was allowed to cool to room temperature. Solid sodium bicarbonate (2 g) was added and the mixture was vigorously stirred for 10 min. Sodium sulfate was added and the mixture was dried. After filtration, the solvent was evaporated and the resulting residue was dried. The product was obtained as a yellow viscous oil (2.27 g, 42% yield) with the following composition determined by GC-MS (Varian 3800/Saturn 2000): diol (4.0%), monoacetate (88.0%), and diacetate (8.0%).

### Comparative Example 5

In a 250 mL round bottom flask, 1,4-cyclohexanediol (3.98 g, 34.3 mmol) was suspended in toluene (100 mL). Acetic acid (2.05 mL, 35.8 mmol) and concentrated sulfuric acid (137 mg, 1.40 mmol) were added and the reaction mixture was heated to reflux temperature. After 4 hours of reflux, the mixture was allowed to cool to room temperature. Solid sodium bicarbonate (2 g) was added and the mixture was vigorously stirred for 15 min. Sodium sulfate was added and the mixture was dried. After filtration, the solvent was evaporated and the resulting residue was dried. The product was obtained as a yellow viscous oil (4.55 g, 84% yield) with the following composition determined by GC-MS (Varian 3800/Saturn 2000): diol (5.5%), monoacetate (76.2%), and diacetate (18.3%).

The above-described examples and comparative examples are summarized in the following table.

| | | | | | % |
|---|---|---|---|---|---|
| Ex. | | Carboxylic | | | Mono- |
| No. | Diol | Acid | System | Catalyst | ester |
| 1 | 1,8-octanediol | acetic | water/ | — | 88.2 |
| | | | toluene | | |
| 2 | 1,8-octanediol | acetic | water/ | H₂SO₄ | 96.8 |
| | | | toluene | | |
| 3 | 1,8-octanediol | acetic | water/ | HCl | 87.8 |
| | | | toluene | | |
| 4 | 1,8-octanediol | acetic | water/ | HNO₃ | 86.0 |
| | | | toluene | | |
| C1 | 1,8-octanediol | acetic | water/ | H₂SO₄ | 53.4 |
| | | | cyclohexane | | |
| C2 | 1,8-octanediol | acetic | toluene | H₂SO₄ | 50.0 |
| 5 | 1,8-octanediol | formic | water/ | H₂SO₄ | 84.1 |
| | | | toluene | | |
| C3 | 1,8-octanediol | benzoic | water/ | H₂SO₄ | 0 |
| | | | toluene | | |
| C4 | 1,8-octanediol | isovaleric | water/ | H₂SO₄ | 0 |
| | | | toluene | | |
| 6 | 1,9-nonanediol | acetic | water/ | H₂SO₄ | 94.3 |
| | | | toluene | | |
| 7 | 1,10-decanediol | acetic | water/ | H₂SO₄ | 75.0 |
| | | | toluene | | |
| 8 | 1,11- | acetic | water/ | H₂SO₄ | 73.0 |
| | undecanediol | | toluene | | |
| 9 | 1,4- | acetic | water/ | H₂SO₄ | 88.0 |
| | cyclohexanediol | | toluene | | |
| C5 | 1,4- | acetic | toluene | H₂SO₄ | 76.2 |
| | cyclohexanediol | | | | |

Various unforeseeable modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

1. A process for preparing monoesters comprising the step of reacting at least one diol with at least one carboxylic acid in a biphasic solvent system, said carboxylic acid being sufficiently water soluble to allow esterification to occur, and said biphasic solvent system comprising water and at least one aprotic solvent in which the resulting monoester has greater solubility than in water.

2. The process of claim 1 wherein said diol is a diprimary or disecondary diol.

3. The process of claim 2 wherein said diol is a diprimary diol.

4. The process of claim 1 wherein said diol is selected from the group consisting of 1,8-octanediol, 1,9-nonanediol, 1,10-decanediol, 1,11-undecanediol, 1,4-cyclohexanediol, and mixtures thereof.

5. The process of claim 4 wherein said diol is selected from the group consisting of 1,8-octanediol, 1,9-nonanediol, 1,11-undecanediol, and mixtures thereof.

6. The process of claim 1 wherein said diol comprises two hydroxyl groups having substantially equal reactivity.

7. The process of claim 1 wherein said diol is symmetric.

8. The process of claim 1 wherein said diol has less than about 14 carbon atoms.

9. The process of claim 1 wherein said carboxylic acid has a solubility in water of at least about 20% by weight at 20° C.

10. The process of claim 9 wherein said carboxylic acid has a solubility in water of at least about 50% by weight at 20° C.

11. The process of claim 10 wherein said carboxylic acid has a solubility in water of about 100% by weight at 20° C.

12. The process of claim 1 wherein said carboxylic acid has a solubility in water greater than or equal to that of isobutyric acid.

13. The process of claim 12 wherein said carboxylic acid is selected from the group consisting of formic acid, acetic acid, trifluoroacetic acid, n-butyric acid, pyruvic acid, propionic acid, and mixtures thereof.

14. The process of claim 13 wherein said carboxylic acid is selected from the group consisting of formic acid, acetic acid, and mixtures thereof.

15. The process of claim 14 wherein said carboxylic acid is acetic acid and the resulting monoester is a monoacetate.

16. The process of claim 15 further comprising the steps of:
(a) oxidizing the remaining hydroxyl group of said monoacetate to form an aldehyde, and
(b) reacting said aldehyde with an alkylidene phosphorane to form the corresponding olefinic monoacetate.

17. The process of claim 1 wherein said aprotic solvent has a polarity index between about 1.5 and about 3.5.

18. The process of claim 17 wherein said aprotic solvent has a polarity index between about 2.0 and about 3.0.

19. The process of claim 1 wherein said aprotic solvent is an aromatic or ether solvent.

20. The process of claim 19 wherein said solvent is selected from the group consisting of toluene, benzene, chlorobenzene, ethylbenzene, xylenes, trifluorotoluene, dichlorobenzene, methyl tert-butyl ether (MTBE), diethyl ether, diisopropyl ether, dibutyl ether, and mixtures thereof.

21. The process of claim 19 wherein said solvent is an aromatic solvent.

22. The process of claim 21 wherein said solvent is toluene.

23. The process of claim 1 wherein said diol and said carboxylic acid are reacted in the presence of an acid catalyst.

24. The process of claim 23 wherein said catalyst is selected from the group consisting of sulfuric acid, nitric acid, hydrochloric acid, and mixtures thereof.
